(19) United States
(12) Patent Application Publication
(10) Pub. No.: US 2013/0230592 A1
(43) Pub. Date: Sep. 5, 2013

(54) NOVEL OMEGA-3 AND OMEGA-6 FATTY ACID COMPOSITIONS AND USES THEREOF

(71) Applicants: Christian TERREAUX, Chardonne (CH); Eleonora HOMBERGER, Aesch (CH); Burkhard KRIWET, Grenzach-Wyhlen (DE); Anne ECKERT, Basel (CH)

(72) Inventors: Christian TERREAUX, Chardonne (CH); Eleonora HOMBERGER, Aesch (CH); Burkhard KRIWET, Grenzach-Wyhlen (DE); Anne ECKERT, Basel (CH)

(21) Appl. No.: 13/862,142

(22) Filed: Apr. 12, 2013

Related U.S. Application Data
(63) Continuation-in-part of application No. PCT/EP2011/067838, filed on Oct. 12, 2011.
(60) Provisional application No. 61/445,736, filed on Feb. 23, 2011.
(60) Provisional application No. 61/392,106, filed on Oct. 12, 2010.

(51) Int. Cl. A61K 31/202; A61K 31/355; A61K 31/714; A61K 31/59; A61K 31/675; A61K 31/519

(52) U.S. Cl. CPC A61K 31/202; A61K 31/675; A61K 31/519; A61K 31/714; A61K 31/59; A61K 31/355
USPC 424/479; 514/52; 424/474; 424/48; 514/89; 424/745; 424/746; 424/769

(57) ABSTRACT

A composition for use in preventing and/or treating and/or reducing organic, including symptomatic, mental disorders, comprising:
a) Docosahexaenoic acid (DHA) and/or the pharmaceutically acceptable derivatives and/or precursors thereof;
b) Eicosapentaenoic acid (EPA) and/or the pharmaceutically acceptable derivatives and/or precursors thereof;
c) γ-linolenic acid (GLA) and/or the pharmaceutically acceptable derivatives and/or precursors thereof; and

at least one vitamin selected from vitamin B6, folic acid, vitamin B12, vitamin E, and/or vitamin D, at least one mineral which is selenium and/or zinc, optionally other vitamins, and optionally a polyphenolic plant extract with antioxidant properties.

### FIELD OF THE INVENTION

The present invention provides novel compositions comprising combinations of omega-3 and omega-6 fatty acids, with at least one vitamin and one or more minerals and an effective amount of polyphenolic plant extract with antioxidant properties. These compositions are particularly useful for treating and/or preventing mental disorders either in healthy subjects or in subjects in need of such treatment.

### BACKGROUND OF THE INVENTION

Degenerative diseases are becoming increasingly important. In Switzerland, Alzheimer's disease affects around 100,000 patients. Patients in the advanced stages of the disease need all-round care, something which has enormous health cost implications and is a huge burden for the patient, their relatives and for society. Today's treatment options are unsatisfactory owing to limited choice, many side effects, high costs and inadequate effect of the treatment. So far, patients can benefit only from a combating of the symptoms and from a deceleration of the course of the disease through drug treatment. The fact that there is a high prevalence of the disease and that only inadequate treatment methods are available shows the necessity and urgency of further research into the area of the pharmacological treatment of Alzheimer's disease and other degenerative conditions.

Extracellular plaques consisting of beta amyloid are a chief characteristic of Alzheimer's dementia and known for independent Reactive oxygen Species (ROS) production and impairment of the mitochondrial function; this leads to an impairment of the cell's energy metabolism and may result in apoptosis. When it comes to the pathogenesis of

Alzheimer's disease, oxidative stress and mitochondrial dysfunction appear to be important factors influencing the course of the illness. Whether oxidative stress and the associated mitochondrial dysfunction are part of the cause or the effect of the illness is so far unclear.

### SUMMARY OF THE INVENTION

The present invention is directed to compositions and methods of treatment that combine the nutritional, health and/or medical benefits provided to subjects by omega-3 fatty acids and omega-6 fatty acids, with nutritional, health and/or medical benefits provided to subjects by selected minerals and vitamins and polyphenolic plant extract with antioxidant properties. The present invention also relates to methods for producing such compositions, and to methods for using such compositions to provide one or more medical benefits to a subject, or to enhance one or more of such benefits in a subject.

The present inventors have surprisingly found this novel composition based on a novel combination of elements, to be highly effective for use in a method of treating and/or preventing mental disorders. In particular the inventors have demonstrated the composition of the invention to have a beneficial synergistic effect in a degenerative disease model.

The present invention thus relates to a composition for use in a method of treating and/or preventing mental and behavioral disorders and/or improving mental health and cognitive functions in a subject, comprising:
a) eicosapentaenoic acid (EPA) and/or the pharmaceutically acceptable derivatives and/or precursors thereof;
b) docosahexaenoic acid (DHA) and/or the pharmaceutically acceptable derivatives and/or precursors thereof;
c) γ-linolenic acid (GLA) and/or the pharmaceutically acceptable derivatives and/or precursors thereof,
   at least one vitamin selected from the group consisting of vitamin B6, folic acid, vitamin B12, vitamin E, and vitamin D, at least one mineral selected from the group consisting of selenium and zinc, and an effective amount of polyphenolic plant extract with antioxidant properties.

The present invention also relates to a composition comprising:
a) eicosapentaenoic acid (EPA) and/or the pharmaceutically acceptable derivatives and/or precursors thereof;
b) docosahexaenoic acid (DHA) and/or the pharmaceutically acceptable derivatives and/or precursors thereof;
c) γ-linolenic acid (GLA) and/or the pharmaceutically acceptable derivatives and/or precursors thereof,
   at least one vitamin selected from the group consisting of vitamin B6, folic acid, vitamin B12, vitamin E, and vitamin D, at least one mineral selected from the group consisting of selenium and zinc, and an effective amount of polyphenolic plant extract with antioxidant properties, for the preparation of a drug for treating and/or preventing mental and behavioral disorders and/or improving mental health and cognitive functions in a subject.

The present invention further relates to a method of treating and/or preventing mental and/or behavioral disorders and/or improving mental health and cognitive functions in a subject, comprising administering to said subject a therapeutically effective amount of a composition comprising:
a) eicosapentaenoic acid (EPA) and/or the pharmaceutically acceptable derivatives and/or precursors thereof;
b) docosahexaenoic acid (DHA) and/or the pharmaceutically acceptable derivatives and/or precursors thereof;
c) γ-linolenic acid (GLA) and/or the pharmaceutically acceptable derivatives and/or precursors thereof,
   at least one vitamin selected from the group consisting of vitamin B6, folic acid, vitamin B12, vitamin E, and vitamin D, at least one mineral selected from the group consisting of selenium and zinc, and an effective amount of polyphenolic plant extract with antioxidant properties.

The compositions according to the present invention are preferably administered orally to a healthy human subject or a human subject in need of such treatment.

Compositions according to the present invention are administered to subjects of at least 50 years old, especially in populations having poor dietary habits for treating and/or preventing symptoms of age associated cognitive decline, symptoms of subjective cognitive impairment, age related cognitive decline (ARCD), age-associated memory impairment (AAMI), mild cognitive impairment (MCI), neurodegenerative disease, central nervous system disturbances, organic, including symptomatic, mental disorders, such as dementia in

Alzheimer's disease, vascular dementia, senile dementia; other degenerative diseases of the nervous system, such as Alzheimer's disease.

According to one aspect, the present invention provides a food supplement comprising the foregoing compositions, and thus can be administered as a food supplement and/or a food for special medical purposes. In another aspect, the present invention provides a pharmaceutical composition comprising the foregoing compositions, specifically said composition is in the form of a pharmaceutical composition, and comprises a pharmaceutically acceptable vehicle.

### DETAILED DESCRIPTION OF THE INVENTION

The present invention provides compositions including unique combinations of omega-3 and omega-6 fatty acids in specific ratios with vitamins, and minerals. In particular, these unique combinations comprise effective amounts of eicosapentaenoic acid (EPA), docosahexaenoic acid (DHA), γ-linolenic acid (GLA), vitamins, and minerals and novel ratios of EPA, DHA and GLA.

The present invention thus provide novel compositions comprising:
a) eicosapentaenoic acid (EPA) and/or the pharmaceutically acceptable derivatives and/or precursors thereof;
b) docosahexaenoic acid (DHA) and/or the pharmaceutically acceptable derivatives and/or precursors thereof;
c) rlinolenic acid (GLA) and/or the pharmaceutically acceptable derivatives and/or precursors thereof,
   at least one vitamin selected from the group consisting of vitamin B6, folic acid, vitamin B12, vitamin E, and vitamin D, at least one mineral selected from the group consisting of selenium and zinc, an effective amount of polyphenolic plant extract with antioxidant properties.

A surprising feature of the present invention is the synergy between the active ingredients of the novel composition. The combination of the components of the novel composition namely DHA, EPA, GLA, polyphenolic plant extract with antioxidant properties, and vitamins and minerals is believed to have a synergistic effect, indicating that the effect on the nutritional status is higher or the effect on improvement of the underlying disease condition is higher than expected from the mere additive effects of the single components. The active ingredients of the composition of this invention have different mechanisms of action thus providing synergistic effects in the treatment and/or prevention of the diseases specified below.

The present invention thus provides synergistic compositions for use in a method of treating and/or preventing mental and/or behavioral disorders and/or improving mental health and cognitive functions in human adults, comprising a) eicosapentaenoic acid (EPA) and/or the pharmaceutically acceptable derivatives and/or precursors thereof; b) docosahexaenoic acid (DHA) and/or the pharmaceutically acceptable derivatives and/or precursors thereof; c) γ-linolenic acid (GLA) and/or the pharmaceutically acceptable derivatives and/or precursors thereof, at least one vitamin selected from the group consisting of vitamin B6, folic acid, vitamin B12, vitamin E, and vitamin D, at least one mineral selected from the group consisting of selenium and zinc, and an effective amount of polyphenolic plant extract with antioxidant properties.

“Fatty acids” refer to a family of carboxylic acids having a hydrocarbon chain of from about 12 to about 24 carbons in length. Unsaturated fatty acids have at least one carbon-carbon double bond in the hydrocarbon chain. Unsaturated fatty acids include monounsaturated fatty acids and polyunsaturated fatty acids (PUFAs). Unsaturated fatty acids are designated by the position of the first double bond from the methyl end of the hydrocarbon chain. Omega-3 fatty acids have a first double bond at the third carbon from the methyl end of the chain; and include, e.g., α-linolenic acid (octadeca-9,12,15-trienoic acid), stearidonic acid (octadeca-6,9,12,15-tetraenoic acid), eicosapentaenoic acid (eicosa-5,8,11,14,17-pentaenoic acid; “EPA”), docosapentaenoic acid (docosa-7,10,13,16,19-pentaenoic acid), eicosatetraenoic acid (eicosa-8,11,14,17-tetraenoic acid), and docosahexaenoic acid (docosa-4,7,10,13,16,19-hexaenoic acid; “DHA”). Omega-6 fatty acids have a first double bond at the sixth carbon from the methyl end of the chain; and include, e.g., linoleic acid (9,12-octadecadienoic acid), γ-linolenic acid (6,9,12-octadecatrienoic acid; GLA), eicosadienoic acid (11,14-eicosadienoic acid), dihomo-γ-linolenic acid (8,11,14-eicosatrienoic acid), arachidonic acid (5,8,11,14-eicosatetraenoic acid), docosadienoic acid (13,16-docosadienoic acid), adrenic acid (7,10,13,16-docosatetraenoic acid), docosapentaenoic acid (4,7,10,13,16-docosapentaenoic acid), and calendic acid (8E,10E,12Z-octadecatrienoic acid), and the like. The present invention also contemplates the use of precursors and derivatives of such fatty acids.

Natural sources such as fish oil are the major sources of omega-3 fatty acids, such as EPA and DHA. There are however other marine source, such as algae and krill. Omega-6 fatty acids may also be obtained from fish oil, but a major source of GLA may be primrose oil, or other vegetable sources, such as borage oil, black current seed oil.

Preferably, the fatty acids according to the present invention may be obtained by extraction, concentration and purification processes starting from fish oils for DHA and EPA, and from primrose oil typically for GLA, as well as by means of semi-synthetic transformation processes, when required.

The fatty acids are derived from fish oil by a refining process:

1. Washing: The crude oil is washed with NaOH to remove free fatty acids and with citric acid to remove water soluble proteins;
2. Winterization: saturated fats are removed by winterization, which comprises a cooling step down to below zero;
3. Bleaching: contaminants are removed by addition of bleaching clay and subsequent filtration;
4. Deodorisation: remaining volatiles which give the fishy taste, are removed, e.g. by steam-washing (the oil is put into near-vacuum and blasted with steam of about 20 atm);
For highly concentrated oils, some additional steps are carried out:
5. Esterification: the triacylglycerols are saponified with alcohol and ethyl esters are formed;
6. Molecular distillation: unwanted fatty acids (short chain FA) are removed by distillation;
7. Re-esterification: triacylglycerols are reconstituted by mixing the ethyl esters with glycerol at elevated temperatures;
The specified concentrations of the individual products are achieved by mixing natural oil with the re-esterified concentrates in an appropriate ratio.

According to the present invention, the relative amounts of the omega-3 fatty acids are preferably in the range of 1 part DHA to 1 to 10 parts (weight per weight (w/w)) EPA, in particular 1 part DHA to 2 to 5 parts (w/w) EPA, such as 1 part DHA to 3 to 3.5 parts (w/w) EPA. The relative amount of the omega-6 fatty acid GLA to the combined omega-3 fatty acids DHA and EPA are preferably in the range of 1 part GLA to 5 to 20 parts (w/w) DHA/EPA, in particular 1 part GLA to 10 to 15 parts (w/w) DHA/EPA, such as 1 part GLA to 11 to 13 parts (w/w) DHA/EPA.

The present synergistic compositions comprise an effective amount of a polyphenolic plant extract with antioxidant properties in a pure or mixed form. Such polyphenolic plant extracts are well known in the art and are describe inter alia Kähkönen MP et al. (J Agric

Food Chem. 1999 October;47(10):3954-62). By way of example and without limitations we may cite green tea extract, which contains as active ingredient epigallocatechin gallate (EGCG).

As indicated above compositions according to the present invention comprise at least one vitamin selected from the group consisting of vitamin B6, folic acid, vitamin B12, vitamin E, and vitamin D. Vitamin B6 is well known in the art as pyridoxine hydrochloride, pyridoxine 5′-phosphate, and pyridoxal 5′-phosphate. Folic acid or of reduced folate includes but is not limited to L-methylfolate; L-5-methyltetrahydrofolate; pteroylmonoglutamic acid; calcium-L-methylfolate; L-5-methyl-tetrahydrofolic acid; (6S) tetrahydrofolic acid; (6S)-5-methyl-tetrahydrofolic acid; 5-methyl-(6S)-tetrahydrofolic acid; 5-formyl-(65)-tetrahydrofolic acid; 10-formyl-(6R)-tetrahydrofolic acid; 5,10-methylene-(6R) tetrahydrofolic acid; 5,10-methenyl-(6R)-tetrahydrofolic acid; 5-formimino-(6S)-tetrahydrofolic acid, and polyglutamyl derivatives of tetrahydrofolate acid). Vitamin B12 corresponds to cyanocobalamin, cobalamin, hydroxocobalam in, 5′-deoxyadenosylcobalamin, and methylcobalamin. Vitamins which may be incorporated in the present compositions may be water-soluble vitamins or oil-soluble vitamins. Preferred compositions of the present invention comprise an effective therapeutically amount of vitamin B6.

The compositions of the present invention may comprise other vitamins such as for example vitamin B1 (thiamin hydrochloride, thiamin mononitrate, thiamine monophosphate chloride, and thiamine pyrophosphate chloride), vitamin B2 (riboflavin and riboflavin 5′-phosphate sodium), niacin (nicotinic acid, nicotinamide, and inositol hexanicotinate), vitamin B5 (panthothenic acid), vitamin H (biotin), vitamin C (ascorbic acid, sodium-L-ascorbate, calcium-L-ascorbate, potassium-L-ascorbate, L-ascorbyl 6-palmitate, magnesium L-ascorbate, and zinc L-ascorbate), vitamin E (D-alpha-tocopherol, DL-alpha-tocopherol, D-alpha-tocopheryl acetate, DL-alpha-tocopheryl acetate, D-alpha-tocopheryl acid succinate, mixed tocopherols, and tocotrienol tocopherol), vitamin A, vitamin D (cholecalciferol and ergocalciferol), panthotenic acid (D-pantothenate, calcium, D-pantothenate, sodium, dexpanthenol, and pantethine), and/or biotin (D-biotin).

These vitamins are incorporated in the compositions of the present invention in amounts so as to provide to the subject the recommended daily allowance (RDA) of vitamins as defined by the WHO and multiples thereof or to provide the reference nutrient Intake (RNI) for vitamins as defined by the Food and Agriculture Organization (FAO) and multiples thereof.

Compositions according to the present invention also comprises at least one mineral selected from the group consisting of selenium and zinc. Sources of zinc may be inter alia zinc oxide, zinc sulfate, zinc lactate, zinc acetate, zinc L-ascorbate, zinc L-aspartate, zinc bisglycinate, zinc chloride, zinc citrate, zinc gluconate, zinc L-lysinate, zinc malate, zinc mono-L-methionine sulphate, zinc oxide, zinc carbonate, zinc L-pidolate, and zinc picolinate). Sources of selenium may be sodium selenate or any other pharmaceutically acceptable form or derivative of selenium, such as L-selenomethionine, selenium enriched yeast, selenious acid, sodium hydrogen selenite, and sodium selenite. Preferred compositions of the present invention comprise a effective therapeutically amount of selenium.

Other minerals which may be incorporated in the synergistic compositions of the present invention. These minerals may be derived from various sources, such as iron source (ferrous sulfate, ferrous fumarate, Fe(III)-hydroxy-polymaltose complex, i.e., Maltofer®, ferrous carbonate, ferrous citrate, ferric ammonium citrate, ferrous gluconate, ferrous fumarate, ferric sodium diphosphate, ferrous lactate, ferric diphosphate, ferric saccharate, ferrous bisglycinate, ferrous L-pidolate, ferrous phosphate, and iron (II) taurate), phosphorus source, magnesium source (magnesium oxide), copper (cupric oxide or any other pharmaceutically acceptable form or derivative of copper, such as cupric carbonate, cupric citrate, cupric gluconate, cupric sulphate, copper L-aspartate, copper bisglycinate, copper lysine complex, and copper (II) oxide), manganese (manganese sulfate or any other pharmaceutically acceptable form or derivative of manganese, such as manganese ascorbate, manganese L-aspartate, manganese bisglycinate, manganese carbonate, manganese chloride, manganese citrate, manganese gluconate, manganese glycerophosphate, manganese pidolate, and manganese sulphate), chromium (chromium chloride or any other pharmaceutically acceptable form or derivative of chromium, such as chromium (III) lactate trihydrate, chromium nitrate, chromium picolinate, and chromium (III) sulphate), molybdenum (sodium molybdate and/or any other pharmaceutically acceptable form or derivative of molybdenum, such as ammonium molybdate: molybdenum VI, potassium molybdate: molybdenum VI, and sodium molybdate: molybdenum VI), fluoride (calcium fluoride, potassium fluoride, sodium fluoride, and sodium monofluorophosphate), iodine, calcium, boron, various mixtures and/or combinations thereof.

The vitamins and/or minerals are, optionally, present in a solid-state form, such as an amorphous powder or a milled material, for example, a finely milled crystalline material, but can also be present in an aqueous or other solution that is a continuous or disperse phase of an emulsion including the one or more edible oils. Water-soluble vitamins and/or minerals present in solid form in compositions of the invention preferably become at least temporarily suspended in, and coated with, the one or more edible oils.

Most preferred synergistic compositions comprise an increased concentration of EPA, DHA, and GLA. More particularly, a preferred ratio of EPA:DHA:GLA is equal to around 9:3:1. Thus the ratio may be around 9:2.5-3.5:0.8-1.2. In one embodiment the ratio is, or is around, 9:2.8-3.2:0.9-1.1.

The compositions of the invention may thus comprise between 30 to 50 mg of EPA, preferably 40 to 45 mg, and most preferably around 42 mg of EPA; between 200 to 400 mg of DHA, preferably between 250 and 350 mg of DHA, preferably around 300mg of DHA; and between 10 to 20 mg of GLA, preferably between 12 to 17 mg of GLA, and most preferably around 15 mg of GLA. The compositions may comprise around 42 mg of EPA, around 300 mg of DHA, and around 15 mg of GLA.

The composition may comprise (in addition to EPA, DHA and GLA) 2, 3, 4, 5 or all of the following: folic acid, vitamin B12, vitamin B6, selenium, zinc, vitamin D3, vitamin E, and

EGCG (epigallocatechin gallate) as the polyphenolic plant substance with antioxidant properties (green tea extract). As a percentage relative to EPA any of the following may be present in the indicated range of amounts:

folic acid: 0.01 to 0.1, preferably 0.03 to 0.07, or around 0.05

vitamin B12: 0.0005 to 0.005, preferably 0.0008 to 0.002, or around 0.001

vitamin B6: 0.05 to 1, preferably 0.1 to 0.5, or around 0.3

selenium: 0.001 to 0.02, preferably 0.004 to 0.12, or around 0.007

zinc: 0.2 to 2, preferably 1 to 1.6, or around 1.4

vitamin D3: 0.0005 to 0.003, preferably 0.001 to 0.002, or around 0.0013

vitamin E: 1 to 4, preferably 1.5 to 3, or around 2

EGCG: 3 to 10, preferably 4 to 8, or around 6.5

Compositions of the present invention may further comprise an antioxidant. The term “antioxidant” as used herein means an agent that can prevent or reduce an oxidation, degradation and/or other decomposition that would otherwise occur to components or ingredients of the compositions of the invention, such as vitamins and/or minerals and/or edible oils. A wide variety of antioxidant agents are commercially available from sources known by those of skill in the art. Other preferred stabilized compositions of the present invention may be obtained by microencapsulation technique, which is a well known technique in the art.

Such antioxidant provides improved stability measured in terms peroxide value and anisidine value and oxidation induction time. Lower stability of EFAs leads to decomposition reactions of fatty acids that form undesirable peroxides and hydroperoxides. The subsequent decomposition of these oxidation products can form volatile and non-volatile aldehydes and/or ketones. The non-volatile components can catalyze further oxidation of the oils and the volatile components give rise to undesirable taste and smell.

Minimizing the amount of oxidation measured by “Peroxide value” (PV) and “Anisidine value” (AV) can have significant implications when assessing the oxidative stability of oil. “Peroxide value” (PV) and “Anisidine value” (AV) as used herein are generally indices of deterioration in edible oil. The peroxide value (PV) is the concentration of peroxide compounds in the oil measured in meq/kg. Peroxide compounds are produced during PUFA oxidation, thus, the higher the value of PV, the more PUFA oxidation that has occurred. The

AV indicates the amount of oxidation that the oil has experienced prior to measurement and is a measure of the concentration of the secondary oxidation products. The AV of oil is a measure of the amount of non-volatile aldehydes and/or ketones in the oil. As the AV of the oil measures the non-volatile aldehyde and/or ketone concentration in the oil (typically, unitless), it is a measure of its oxidative history. Aldehydes and ketones are produced from the decomposition of the peroxide or hydroperoxide species, which are primary oxidation products of the olefinic functionality on a fatty acid. Methods for measuring PV or AV of an oil are well known in the art.

Other antioxidants for increase the stability of the oily combination may include for example at least one vitamin E isoform, tocopherols or any other agent that reduces oxidative stress. By way of example of antioxidants we may cite tocopherols (4 toco), origanox®, oils extracts from several spices, such as oregano (Origanum vulgare), thyme (Thymus vulgaris), rosemary (Rosmarinus officinalis) or rosemary extract, sage (Salvia officinalis), and clove (Syzygium aromaticum). Origanox® which is commercialized by Harrington Nutritional, is a natural, completely water soluble, powerful antioxidant, extracted from edible herb species, belonging to the Labitae family (such as Origanum vulgare and Salvia officinalis) and considered as GRAS by the FDA. Origanox® is offered in various standardized grades of antioxidant activity. Alternatively, such antioxidant may include vitamin C, synthetic antioxidants, e.g., BHT, TBHQ, ethoxyquin, alkyl gallates, hydroquinones, tocotrienols), NXY-059 (Disufenton sodium); chain-breaking phenolic antioxidants (such as Vitamin E and butylated hydroxytoluene [BHT]); phenyl-substituted nitrones; azulenyl-substituted nitrones; α-phenyl-N-tert-butyl nitrone; stilbazulenyl nitrone, polyphenolic plant extract with antioxidant properties etc. Preferred antioxidant is Origanox® OS-F commercialized by Frutarom. Origanox® OS-F is an oil dispersible extract from the edible herb specie Melissa Officinalis and Sunflower-Oil, dispersed with PGPR (Polyglycerol polyricinoleate) and acidified ascorbic acid. Another preferred antioxidant may be rosemary (Rosmarinus officinalis) or rosemary extract. Most preferably, the OXY'LESS® CLEAR commercialized by Naturex is used in the compositions of the present invention. OXY'LESS® CLEAR corresponds to an extract obtained from rosemary leaves or Rosmarinus officinalis L.

Synergistic compositions according to the present invention are thus particularly useful in a method of treating and/or preventing mental and/or behavioral disorders and/or improving mental health and cognitive functions in human adult subjects. The present invention thus also relates to the use of the compositions as defined above in the manufacture of a drug for the treatment and/or the prevention of mental and/or behavioral disorders and/or improving mental health and cognitive functions in human adult subjects.

The present invention further relates to methods of treating and/or preventing mental and/or behavioral disorders and/or improving mental health and cognitive functions of human adult subjects, comprising administering a therapeutically effective amount of the above compositions to human adult subjects.

The terms “subject” and “patient” used interchangeably herein, refer to a mammal, a human. Such human subject herein includes adults and seniors, either normal or mainstream and/or with underlying disease conditions. Adult human subjects according to the invention generally refer to subjects having at least 50 years old. Seniors subjects refer to human subjects above 60 or 65 years old. The subject may have or be at risk of any of the conditions mentioned herein. The subject may have been diagnosed with any of the conditions mentioned herein. In one embodiment the subject has a specified condition as mentioned herein, and does not suffer from any other conditions.

It is believed that such combinations of omega-3 and omega-6 fatty acids with specific vitamins and minerals provide adult and senior subjects, either normal or mainstream and/or with underlying disease conditions with a superior heath benefit in terms of mental health and cognitive functions.

“Cognitive functions” or “Signs of cognitive performance” or “symptoms of cognitive dysfunction” as used herein may be selected from a group comprising fine motor skills (like cutting, writing etc . . . ), gross motor skills (running, jumping etc.,), motor coordination abilities (e.g., hand-eye coordination, like holding a pencil or buttoning a shirt etc . . . ), learning abilities (like sequencing, memory, organization etc.,), language-based learning abilities, reading abilities (understanding the relationship between sounds, letters and words, letter and word recognition, understanding words and ideas, reading speed and fluency, general vocabulary skills etc . . . ), writing abilities (neatness and consistency of writing, accurately copying letters and words, spelling consistency, writing organization and coherence etc . . . ), auditory processing skills, visual perception skills, social and emotional skills, ability to complete task, ability to stay on task, ability to follow instructions, ability to complete assignments, long term memory, short term memory, ability to make a decision, ability to follow through on decision, ability to engage in conversations, sensitivity to surroundings, ability to plan, ability to carry out plan, ability to listen, interruptions in social situations, temper tantrums, level/frequency of frustration, level/frequency restlessness, frequency/level fidgeting, ability to exhibit delayed gratification, aggressiveness, demanding behavior/frequency of demanding behavior, sleep patterns, restive sleep, interrupted sleep, awakening behavior, disruptive behavior, ability to exhibit control in social situations, ability to extrapolate information and ability to integrate information etc . . .

As used herein, the terms “treatment and/or prevention” “treating and/or preventing” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) reducing the incidence and/or risk of relapse of the disease during a symptom-free period; (b) relieving or reducing a symptom of the disease; (c) preventing the disease from occurring in a subject which may be predisposed to the disease but has not yet been diagnosed as having it; (d) inhibiting the disease, i.e., arresting its development (e.g., reducing the rate of disease progression); (e) reducing the frequency of episodes of the disease; and (f) relieving the disease, i.e., causing regression of the disease.

In particular, the compositions of the present invention are believed to be particularly effective:

for administration to a healthy human subject or a human subject in need of such treatment;

for use in a method of preventing and/or treating and/or reducing symptoms of age associated cognitive decline, symptoms of subjective cognitive impairment, age related cognitive decline (ARCD), age associated memory impairment (AAMI), mild cognitive impairment, neurodegenerative disease, central nervous system disturbances, Alzheimer's disease, dementia in a subject of at least 50 years old.

Especially for treatment of abnormalities related to the central nervous system, although therapeutic interventions have been used to address individual aspects of impaired neurological function, it is desirable to provide a comprehensive holistic approach for the treatment. When complex, interrelated disease pathways are involved in an abnormal physiologic condition, a single agent does not provide significant beneficial action. Multiple sites of intervention are mandatory. It is an important feature of the invention that the components act together to provide a synergistic effect by effecting different pathways of action.

The present invention relates to compositions as defined above for use in preventing and/or treating and/or reducing organic, including symptomatic, mental disorders, such as dementia in Alzheimer's disease, vascular dementia, senile dementia; other degenerative diseases of the nervous system, such as Alzheimer's disease; or symptoms of age associated cognitive decline; symptoms of subjective cognitive impairment; age related cognitive decline (ARCD); age associated memory impairment (AAMI); mild cognitive impairment in a subject of at least 50 years old.

These are administered to subjects of at least 50 years old in need either as food supplement or as pharmaceutical formulations.

“Symptoms of age associated cognitive decline” are selected from the group consisting of remembering names, remembering numbers, recalling location of objects, remembering specific facts, inability to concentrate, confusion, hallucinations and delusions, altered sensation or perception, impaired recognition (agnosia), aphasia, altered sleep patterns, motor system impairment, impaired skilled motor function, disorientation, memory deficit, absent or impaired language ability, personality changes, behavioral change, lack of spontaneity and deterioration of musculature and mobility.

Mild Cognitive Impairment is defined as objective memory impairment below an age-appropriate norm on the Rey Auditory Verbal Learning Test, Selective Reminding Test, the Weschler Logical Memory Test or other objective cognitive test.

As used herein, the terms “Alzheimer's disease” are to be taken to include both Alzheimer's dementia and Alzheimer's sclerosis. Alzheimer's disease is a progressive neurocognitive disorder which, without preventive intervention, may affect 10% of the developed world's population. Susceptibility to the disease is strongly influenced by genes.

Two forms of this disease have been attributed to mutations on chromosomes 14 and 21 that act as dominant genetic traits. At least two additional forms of Alzheimer's disease are presumed to be provoked by mutations or polymorphisms located elsewhere in the genome. The symptoms of Alzheimer's disease appear typically between ages 65 and 90. Symptoms include deterioration of cognition, memory and language.

Dementia may be dementia of the Alzheimer's type with early onset uncomplicated, dementia of the Alzheimer's type with early onset with delusions, dementia of the Alzheimer's type with early onset with depressed mood, dementia of the Alzheimer's type with late onset uncomplicated, dementia of the Alzheimer's type with late onset with delusions and dementia of the Alzheimer's type with late onset with depressed mood.

According to one aspect, the present invention provides a food supplement comprising the foregoing compositions, and thus can be administered as a food supplement and/or a food supplement for special medical purpose. In another other aspect, the present invention provides a pharmaceutical composition comprising the foregoing composition; specifically said composition is in the form of a pharmaceutical composition, further comprising a pharmaceutically acceptable vehicle or carrier. In this embodiment precursors of the substances are given to subject.

As used herein, “pharmaceutically acceptable carrier” or “pharmaceutically acceptable vehicle” includes any material which, when combined with an active ingredient of a composition, allows the ingredient to retain biological activity and without causing disruptive reactions with the subject's immune system. Examples include, but are not limited to, any of the standard pharmaceutical carriers such as a phosphate buffered saline solution, water, emulsions such as oil/water emulsion, and various types of oils and lipophilic solvents. Compositions comprising such carriers are formulated by well known conventional methods (see, for example, Remington's Pharmaceutical Sciences, Chapter 43, 14th Ed. or latest edition, Mack Publishing Co., Easton Pa. 18042, USA; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc. Typically any of the substances mentioned herein are administered in isolated or purified form.

Both synergistic stabilized food supplement and synergistic stabilized pharmaceutical compositions according to the present invention are preferably in a form suitable for oral consumption.

By way of example, oral synergistic stable food supplement or pharmaceutical compositions may be in the form of hard or soft capsule, microcapsule, oil, syrup, suspension, solid or semi-solid, tablet, sugar-coated tablets, powder, pellet, film-coated tablet, granule, emulsion, paste, gels, lozenges, gums, or chewable formulation, such as chewing-gum. These oral formulations are preferably gastro-resistant formulations.

Most preferably, such compositions are in the form either of hard capsules of gelatin, or soft gel capsule which present a one-piece, hermetically sealed soft gelatin shell that is filled with a material, substance or compositions (a “fill”). The soft gel shell is generally comprised of a film-forming material, such as gelatin, for example type A and/or type B, and water-dispersible or water-soluble plasticizers (to impart flexibility). Soft gel capsules may be produced using known methods and conventional machinery. The size of the capsules is preferably so chosen that the preferred daily dose is provided by 1, 2, 3, 6 or 9 capsules.

The present composition may also include other pharmaceutically acceptable carriers and desirable additives such as, without limitation, starches, sugars, fats, amino acids, proteins, derivatives thereof or combinations thereof. Inclusions of additives which assist in formulating the final composition are also desirable. In one embodiment the administered composition comprises as active ingredients only the specified substances and no other substances. In another embodiment the composition which is administered comprises only 4, 5 or 6 to 10 active ingredients.

Alternatively, the compositions of the present invention combination may be administered as an oil to be taken in liquid form or as an oily suspension, for example added to normal food. Preferably such oil contains further fat-soluble flavors in order to change or mask the fishy taste of the combination. When administered as syrup the combination is provided as an emulsion in water containing emulsifying compounds and suitable flavors. Oils, oily suspensions or syrups are preferably provided in containers allowing simple dosing.

Pharmaceutical or food supplement dosage forms may contain excipients. Excipients include fillers, dyes or pigments, flavors, stabilizers, extenders, binders, humidifiers, surfactants, lubricants, and the like. Excipients must be of sufficiently high purity and sufficiently low toxicity to render them suitable for administration to the subject being treated. An excipient can be inert or it can possess pharmaceutical benefits. Excipients are selected with respect to the intended form of administration, e.g. oral tablets, capsules, powders, syrups, emulsions, suspensions, and the like, and consistent with conventional pharmaceutical practices. For example, for oral administration in the form of gel capsule the omega-3 fatty acid formulation may be combined with filler, a preservative, flavorant, colorant or the like.

Stabilizers may be added to improve the stability of EFAs. While the relationship of the concentration of stabilizers to the rate of fatty acid oxidation depends on the specific stabilizer, this relationship can be complicated by the presence of more than one stabilizer. The addition of multiple stabilizers can act to stabilize each other and when this occurs, a combination of two or more stabilizers can be more effective at terminating free radicals than a single stabilizer or at the same time the different stabilizers may antagonize each other's activities. Hence optimizing the right stabilizer combinations to improve stability of the EFA formulation is not a matter of routine experimentation. The present inventors have invented the correct combination of elements which makes the formulations of the invention unique in their combination and stability.

The present composition is advantageously formulated into a convenient dosage form, such as a tablet or a film coated tablet, or enclosed in a shell or capsule. Also included herein are pharmaceutical compositions, comprising pharmaceutical formulations in a unit dosage form. In such dosage forms, the formulation is subdivided into suitably sized unit doses containing appropriate quantities of the omega-3 fatty acids, an effective amount to achieve the desired purpose. Formulation of the desired ingredients, nutrients and/or additives may be accomplished by conventional methods. Advantageously, the final composition is encapsulated in a capsule, such as a gelatin capsule, and more advantageously, a soft-shell gelatin capsule. For example, compositions including the vitamins, minerals, EPA DHA and GLA in desired forms and quantities, can be made by weighing the individual ingredients, and blending them together to form a homogeneous mixture for final formulation or encapsulation. Other oral formulations within the scope of the invention includes microcapsule, suspension, solid or semi-solid, tablet, sugar-coated tablets, powder, pellet, film-coated tablet, granule, emulsion, paste, gels, chewable formulation, gastro-resistant formulation, extended release formulations, sustained release formulations, extended release formulations, intermediate release formulations, oral formulations with phase release, like monophasic, biphasic release and the like.

Packaged pharmaceutical formulations are included herein. The invention includes providing prescribing information, over the counter medical use information, or nutritional information for the dosage form, for example, to a patient or health care provider, or as a label in a packaged pharmaceutical formulation. Information included in the pharmaceutical package may include for example efficacy, dosage and administration, contraindication and adverse reaction information pertaining to the dosage form. The formulations of the present invention may also be provided in combination with vitamins or mineral supplements either in a single unit dosage form or in separate unit dosage forms. In one embodiment the invention provides a method of preventing or treating a subject comprising first carrying out a diagnostic test for any of the conditions mentioned herein and if the subject is found to be at risk of the condition or if the subject has the condition, administering to the subject the composition of the invention.

The different components of the composition of the invention can be administered as part of the same composition or separately to the subject. Thus the invention provides a product for use in preventing and/or treating and/or reducing organic, including symptomatic, mental disorders, comprising:
a) Docosahexaenoic acid (DHA) and/or the pharmaceutically acceptable derivatives and/or precursors thereof;
b) Eicosapentaenoic acid (EPA) and/or the pharmaceutically acceptable derivatives and/or precursors thereof;
c) γ-linolenic acid (GLA) and/or the pharmaceutically acceptable derivatives and/or precursors thereof; and
   at least one vitamin selected from the group consisting of vitamin B6, folic acid, vitamin B12, vitamin E, and vitamin D, and at least one mineral selected from the group consisting of selenium and zinc, and optionally other vitamins, and optionally polyphenolic plant extract with antioxidant properties in a pure or mixed form; for simultaneous, separate or sequential administration.

### EXAMPLES

### Example 1

### Example 1.1 In Vitro Activity on Neuronal Cell Viability and Growth

Primary neuronal cells subjected to oxidative stress, i.e., by addition of inflammatory factors, such as LPS, or by addition of beta amyloid, are used as an in vitro model of experiment to assess the impact of the composition according to the invention on neuronal cells.
Primary stressed neuronal cells receive (1) the mixture of EPA, DHA, and GLA; (2) vitamin B6, folic acid, vitamin B12, vitamin D, vitamin E, selenium and zinc; (3) polyphenolic plant extract with antioxidant properties (for ex. green tea extract); (4) the mixture of (1), (2), and (³).
Neuronal cell viability and neuronal growth is then assessed by the formation of neuritis and synaptic markers.

### Example 1.2 In Vitro Activity on Stressed Neuronal Cells

An in vitro model of rat cortical and hippocampal neuronal cells subjected to mechanical and/or hypoxic injuries as described by Gladman S J, et al., (Neuroscience (2010), doi: 10.1016/j.neuroscience.2010.07.009). Alternatively, rat cortical and hippocampal neuronal cells are subject to neuro-inflammation through addition of lipopolysaccharide (LPS) beta-amyloid protein.
Impact of the composition of the present invention on the prevention and/or treatment of neuroinflammation and neurodegeneration is then assessed using (1) the mixture of EPA, DHA, and GLA; (2) vitamin B6, folic acid, vitamin B12, vitamin D, vitamin E, selenium and zinc; (3) polyphenolic plant extract with antioxidant properties (for ex. green tea extract); (4) the mixture of (1), (2), and (3).

### Example 1.3 In Vitro Activity on Astrocytes

Grintal B, Champeil-Potokar G, Lavialle M, Vancassel S, Breton S, Denis I. 2009. Inhibition of astroglial glutamate transport by polyunsaturated fatty acids: evidence for a signalling role of docosahexaenoic acid. Neurochem. Int.54: 535-543
In vitro astrocytes cultures from neonate and adult rat cortex are used to assess cognition related mechanisms:

Astroglial glutamate transport (activity and expression of transporters), synthesis of D-serine, morphological plasticity

Activation of the PLA2 axis and production of S100B protein.

Cultures grow in presence of IL1beta, a characteristic cytokine of aging brain inflammation, in order to mimic conditions of neuronal aging.
The efficacy is assessed using (1) the mixture of EPA, DHA, and GLA; (2) vitamin B6, folic acid, vitamin B12, vitamin D, vitamin E, selenium and zinc; (3) polyphenolic plant extract with antioxidant properties (for ex. green tea extract); (4) the mixture of (1), (2), and (3).

### Example 1.4 Effect of the Compositions of the Present Invention on Aged Rats with Cognitive Decline

Aged wild type rats (over 3 years old) are used as an in vivo model for assessing the impacts of the composition according to the present invention on senior with cognitive decline.
Tested rats are divided in several groups which receive (1) the mixture of EPA, DHA, and GLA; (2) vitamin B6, folic acid, vitamin B12, vitamin D, vitamin E, selenium and zinc; (3) polyphenolic plant extract with antioxidant properties (for ex. green tea extract); (4) the mixture of (1), (2), and (3).
Both treated and placebo groups are assessed by the following experiments: (i) behavioral experiments, and (ii) biochemical experiments, e.g., measure of the status of fatty acid in membranes of erythrocytes and neurons.

### Example 1.5 Treatment of Senior Subjects with Subjective Cognitive Impairment

The composition according to the present invention comprising the mixture of the mixture of EPA, DHA, and GLA; (2) vitamin B6, folic acid, vitamin B12, vitamin D, vitamin E, selenium and zinc is administered as a food supplement to elderly subjects with subjective cognitive impairment or mild cognitive impairment. In addition to this nutritional program, the subjects are also submitted to a physical activity program.
Subjective cognitive impairment (SCI) has been showed as being indicative of mild cognitive impairment (MCI) and an early stage of pathological cognitive deficit, e.g., 15 years before the diagnosis of Alzheimer's disease, vascular dementia or other dementia illness.
Effects of a nutritional supplementation with the composition of the present invention alone or in combination with a physical activity program are thus assessed on elderly patients with SCI or MCI.
The randomized, multi-centre, placebo-controlled study is performed on individuals over 55 years old with spontaneous cognitive (memory) complaints during 12 months.
Impacts of interventions on cognitive behavior and clinical symptoms are determined by assessing cognition in a computerized test battery (attention, memory, speed of reaction).
Secondary endpoints are included:

Cardiovascular parameters: blood lipids, HR, HRV, aerobic fitness,

Blood markers: PUFAs, nutrients, plasma antioxidant capacity,

Different domains of cognition,

Behavior and social behavior,

Quality of life,

Mood and anxiety, and

Imaging (MRI, fMRI, SPECT) in subgroups.

### Example 2 Effects of Fish Oil, Polyphenolic Plant Extract with Antioxidant Properties (Green Tea Extract), Vitamins and Minerals on Mitochondrial Function in an Alzheimer's Cell Culture Model

The work described in this Example aimed to determine the effect of fish oil, of polyphenolic plant extract with antioxidant properties (green tea extract) and a multivitamin and mineral mixture (MVM), administered individually or in combination, on mitochondrial function. In addition it examined the ability of substances and combinations to protect the mitochondria from oxidative stress.
These effects were investigated after a 24-hour substance treatment and after a treatment period of 7 days' duration; this was achieved by determining the metabolic activity of the mitochondria using the MTT assay and by determining the cellular energy level using the ATP bioluminescence assay.
The experiments were carried out on stably transfected human neuroblastoma cells (SH-SY5Y). Cells transfected with human wild type APP expressed an increased amount of APP which lead to excess production of beta amyloid; they therefore served as an Alzheimer cell culture model (APP cells). This transfection made it possible to study the effect of chronic beta amyloid stress and the resulting altered response to different substances. MOCK cells transfected with the appropriate empty vector served as a control in order to investigate general effects on mitochondrial function.
Previous results from the work group showed increased MTT reduction after three hours of 0.5 mM H2O2 induced stress in MOCK and APP cells that were treated for 24 hours with a fish oil (EPA-DHA ratio 2:1; 10 μM EPA and 5 μM DHA). In another study, a treatment over seven days with another fish oil (EPA-DHA ratio 3:1; 300 μM EPA, 100 μM DHA, 30 μM GLA) produced a significant increase in the ATP level in APP cells and a less pronounced one in MOCK cells. Also a protective effect was shown following a secondary insult (0.5 mM H2O2). This work built on the results of both previous studies in order to supplement their data.

### MTT Assay

The MTT assay which is a quantitative colorimetric method for the non-radioactive quantitation of cell proliferation and cell viability was used. The assay is based on the cleavage of the yellow tetrazolium salt 3-(4,5-Dimethylthiazol-2-yl)-2,5-diphenyl tetrazolium bromide (MTT) into the purple formazan crystal by mitochondrial succinate dehydrogenase (SDH). SDH is a membrane-bound protein that forms part of the citric acid cycle where it is important for the oxidation of succinate into fumarate. It simultaneously forms Complex II in the electron transport chain in the mitochondria and with the reduction equivalent FADH2 produced from the succinate oxidation reduces ubiquinone into ubiquinol which then moves further to Complex III.

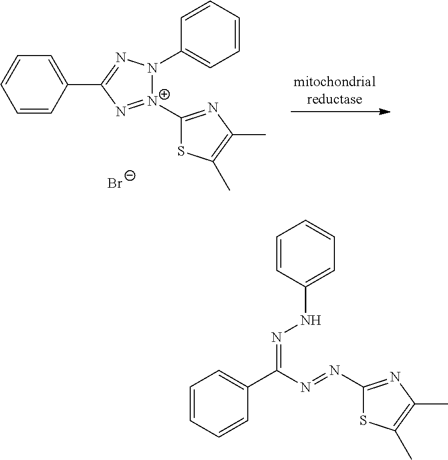 [SMILES: Cc1nc(-[n+]2nc(-c3ccccc3)nn2-c2ccccc2)sc1C ; [Br-] ; Cc1nc(/N=N/C(=N\Nc2ccccc2)c2ccccc2)sc1C]

The conversion of MTT is proportional to the mitochondrial dehydrogenase activity. This cellular reduction involves the pyridine nucleotide cofactors NADH and NADPH. Since MTT cleavage only takes place in metabolically active cells, there exists a direct correlation between the number of living cells and the quantity of purple formazan crystals formed.

Since the crystals formed precipitate out in an aqueous environment they must be dissolved by adding a solvent. This produces a coloured solution whose absorption can be determined using a spectrophotometer at a wavelength of 595 nm. Here the absorption correlated with the cell viability and cell proliferation. Absorption values for treated cells that are higher than the values for untreated cells indicate increased cell proliferation, whilst lower values go hand in hand with reduced cell proliferation or cell viability.

It should be noted that some chemicals or phytochemicals interact directly with MTT or can bring about changes in the succinate dehydrogenase activity and hence lead to changed MTT reduction even though the number of cells remains the same. Bearing the latter influential factors in mind, this text referred to the metabolic activity of the mitochondria as opposed to the cell viability and cell proliferation.

### Method

The Cell Proliferation Kit I (MTT) made by Roche was used. Since the objective was to compare the metabolic activity of MOCK and APP cells and since the quantity of formazan crystal formed correlated with the number of cells, the cells were, after trypsinization and centrifuging, counted out using a Neubauer chamber and subsequently diluted to the same cell number. Using a transparent 96-well plate 2.5*104 cells were seeded per well (100 μL), and the plate then incubated for 24 hours at 37° C., 6.5% CO2. After 24 hours the cells were stressed with 0.5 mM H2O2; this involved pipetting 5 μL of a 10 mM H2O2 solution into each well, which corresponds to a dilution of 1:20. After an hour in the incubator, 10 μL (5 mg/mL) of MTT solution was pipetted into each well, which corresponds to an end concentration of 0.5 mg/mL in the well, and the cells were incubated again for two hours. The MTT was converted by the cells into the purple formazan crystals which slowly began to precipitate out and became visible under the microscope or to the naked eye. After a total of three hours of H2O2 stress the MTT conversion reaction was stopped by adding 100 μL solution buffer to each well; this lysed the cells and dissolved the formazan crystals. The plate was left to stand in the incubator overnight. The following morning the absorption of the coloured solution was determined at a wavelength of 595 nm on the Victor spectrometer.
The MTT assay was, for each substance (combination), carried out on at least two different days (respectively with two different cell passages) and at least two plates were seeded simultaneously each day.

### Test Substances

The present work aimed to determine the effect of fish oil, green tea extract and a multivitamin and mineral mixture (MVM), administered individually or in combination, on mitochondrial function. The quantities of substances in the combination product for oral administration were based on recommended daily dosages. After oral intake of 750 mg EPA a plasma level of about 417 μmol/L was reached. In earlier studies by the research group, an end concentration of 300 μM was tested, which corresponds to an administered quantity of about 538.5 mg/d. To be able to compare the data later, we worked with an end concentration of 300 μM (=0.090738 mg EPA/mL). In order to maintain the ratios of the substance quantities in the combination product, the percentage proportions of the other substances relative to EPA was calculated and the substance quantities standardised to the EPA plasma level. Table 1 lists the percentage proportions of the individual substances relative to EPA along with the desired end concentrations. The products were not pure substances and only contained a certain proportion of the test substances.

After the substances had been dissolved in a suitable solvent, it was possible to produce stock solutions which were then aliquoted and stored at −20° C. Shortly before the start of the MTT or ATP assays the stock solutions were diluted with DMEM and fresh working solutions were produced which could then be used immediately.
The working solutions were each first vortexed well with the medium in a tube and then the cell suspension was added; they were then homogenised and distributed onto the 96-well plates or into Petri dishes. The concentration of the working solution in the well should ideally be below 10% and be not higher than 20%. For the assays with the individual substances and the combinations, the following quantities of working solution were used in the well:
Assays with Individual Substances:
Fish oil: 10% fish oil WS in cell suspension
Green tea: 10% green tea WS in cell suspension
Vitamins and minerals: 7% vitamin and mineral mixture working solution in cell suspension respectively 1% of the individual vitamin and mineral working solutions
Assays with Combinations:
Fish oil+green tea: 5% fish oil WS+5% green tea WS
Fish oil+vitamins and minerals: 5% fish oil WS+7% vitamin and mineral mixture WS
Fish oil+green tea and vitamins and minerals: 5% fish oils WS+7% vitamins and minerals mixture WS
+5% green tea WS

### Substance Treatment of the Cell Culture

Both neuroblastoma cell lines (SH-SY5Y) MOCK and APP were treated for 24 hours and for 7 days with the individual substances and with combinations of these substances. After the 24-hour treatment and the 7-day treatment the treatment effect was studied using MTT and ATP assays. The 24-hour and 7-day treatment are explained in further detail in the next two sections.

### 24-Hour Treatment

On the first day of the 24-hour treatment the cells were counted, treated with the substances and seeded.
To achieve this, the stock solutions were first brought to the desired concentration of the working solutions with DMEM.
After the cells have been counted, they were diluted to the desired number of cells with DMEM and with the working solutions to 4 mL. To do this it was first necessary to mix the medium well with the working solutions in test tubes and subsequently to add the necessary quantity of cell suspension. The working solutions were additionally diluted through this process, something which was taken into account in the calculations. The solutions in the test tubes could now be transferred to the channels of two channelled trays to enable the filling of the plates with a multipipette from here. The cells were incubated for 24 hours at a CO2 content of 6.5% and 37° C.
On the second day some of the cells were stressed with 0.5 mM H2O2 and the assays are carried out. On the third day only the photometric measurement on the spectrometer for the MTT assay was carried out.

### 7-Day Treatment

Both the neuroblastoma cell lines were treated for seven full days with the individual substances or with combinations of the substances in order, at the end of the treatment, to be examined with ATP and MTT assays.
On the first day of the treatment for seven full days one Petri dish of MOCK cells and one Petri dish of APP cells were split 1:4 and three of each of these treated with substances and one Petri dish left untreated as a control in each case. The stock solutions were first diluted with DMEM in order to obtain working solutions of suitable concentrations. The working solutions were mixed well with the cell culture medium in a test tube and then made up to 10 mL with 1 mL cell suspension. The working solutions were additionally diluted through this process, something which was taken into account in the calculations. The cell culture dishes were placed back in the incubator (6.5% CO2, 37° C.) and left to stand there for three full days.
On the fourth day the cells were split and treated again with the substances. In principle the same procedure was followed as for the first day except that the cells were now split 1:3 and two dishes of each were retained and treated. The cells were incubated once again for three days.
On the seventh day the cells were, after six days of treatment, be diluted, and seeded into 96-well MTT and ATP plates. Here too, the cells were treated once again with the substances so as to achieve, after 24 hours of incubation, a total treatment duration of seven days. From the seventh day the same procedure applies as for the 24-hour treatment.

### Statistical Analysis

GraphPadPrism 5®-Software was used for the statistical analysis and graphical representation of the experimental results. The histograms presented the averages of the results and also showed their standard error. Statistically significant differences were determined using the Student's t-Test. The paired t-Test was used for the effects of substance treatment and the unpaired t-Test was used for the H2O2 effect. P values of less than 0.05 (*), 0.01 (**) or 0.001 (***) were deemed to be statistically significant.

### Results

This work examined the effect of a variety of food supplements (fish oil, EGCG (epigallocatechin gallate) from green tea, Vitamin D, E, B6, B9, B12, selenium and zinc) on mitochondrial function in vitro assays. It particularly focused on the protective effects on the mitochondria following oxidative stress (0.5 mM H2O2).
The role played by reactive oxygen species and oxidative stress in the pathogenic cascade of Alzheimer's disease is generally acknowledged thanks to the experimental data available and human studies. As a result of the accumulation of oxidative damage and impairment of the antioxidative systems in older people, oxidative stress becomes a major feature of the ageing process. ROS disrupt and damage the cell metabolism through the oxidation of membrane lipids, proteins and nucleic acids. The amyloid plaques characteristic of Alzheimer's are themselves involved in ROS production and can lead to mitochondrial dysfunction, as a result of which the energy balance of the cells is disturbed and apoptosis may occur. Mitochondria, being the powerhouses of the cells, have a central role and in particular are essential in the highly metabolically active neurons. They are the main centres of production for ROS and hence simultaneously targets for oxidative stress. Since mitochondrial dysfunction may accompany severe neurodegenerative diseases, this study examined different substances for their protective effects on the mitochondrial metabolism basally and after induced stress.
The mitochondrial function was determined after a 24-hour and a 7-day treatment with fish oil (300 μM EPA, 87 μM DHA, 35 μM GLA), EGCG (13 μM) or MVM ‘cocktail’, respectively with different combinations of the substances, using an MTT assay in order to establish the mitochondrial metabolic activity. The assays were carried out on human neuroblastoma cells (SH-SY5Y). The MOCK cells transfected with an empty vector serve as a control. APP cells, which are stably transfected with the human wild type APP and, thanks to the increased APP expression contain an excess production of amyloid beta, serve as an Alzheimer's cell culture model.
Previous work shows that APP cells are exposed to an Aβ burden that is greater than for MOCK cells by a factor of three, and that ATP synthesis in APP cells is significantly impaired compared with the control cells and attributes this to Aβ-induced damage to Complex IV. The damage in terms of cellular energy level was confirmed by the assays carried out in this study. The assays also showed that APP and MOCK cells have the ability to compensate for H2O2-induced stress after a certain amount of time, with the cell's own protective mechanisms functioning better in MOCK cells than in APP cells.
The results of the effects of substances on the mitochondrial function have been shown in Table 2. The substance combinations stimulated the mitochondria more strongly than the individual substances and that longer treatment duration was required (7 days as opposed to 24 hours) for the protective mechanisms of the substances to develop. The effects observed after a longer exposure of the cells were in line with the expected in vivo situation: effects of the individual substances in elderly populations occurred after a longer term supplementation and not following acute administration.
The synergistic effect of the whole combination was shown by the higher protective effect (APP cells, 7 days, 3h H₂O₂ exposure; +57.8%, p=0.0002) compared to the sum the respective 2 by 2 combinations (EPA/DHA/GLA+EGCG; +6.3%, p=0.0121 and EPA/DHA/GLA+MVM; +42.4%, p=0.0001). This was also demonstrated for the same cell line under control conditions.

TABLE 1
Proportions of the individual substances relative to EPA content

| Combination | | | % proportion | End |
|---|---|---|---|---|
| product | | | relative to EPA | concentration mg/mL |
| EPA | 750 | mg | 100 | 0.090738 = 300 μM |
| DHA | 238 | mg | 31.74 | 0.028523 = 87 μM |
| GLA | 81 | mg | 10.75 | 0.0097 = 35 μM |
| Folic acid | 400 | μg | 0.053333333 | 4.8394E−05 = 109 nM |
| Vitamin B12 | 7.5 | μg | 0.001 | 9.0738E−07 = 575 pM |
| Vitamin B6 | 2 | mg | 0.266666667 | 0.00024197 = 1.4 μM |
| Selenium | 55 | μg | 0.007333333 | 6.6541E−06 = 84 nM |
| Zinc | 10 | mg | 1.333333333 | 0.00120984 = 18 μM |
| Vitamin D3 | 10 | μg | 0.001333333 | 1.2098E−06 = 3 nM |
| mixed tocopherol | 15 | mg | 2 | 0.00181476 |
| EGCG | 50 | mg | 6.666666667 | 0.0060492 = 13 μM |

TABLE 2

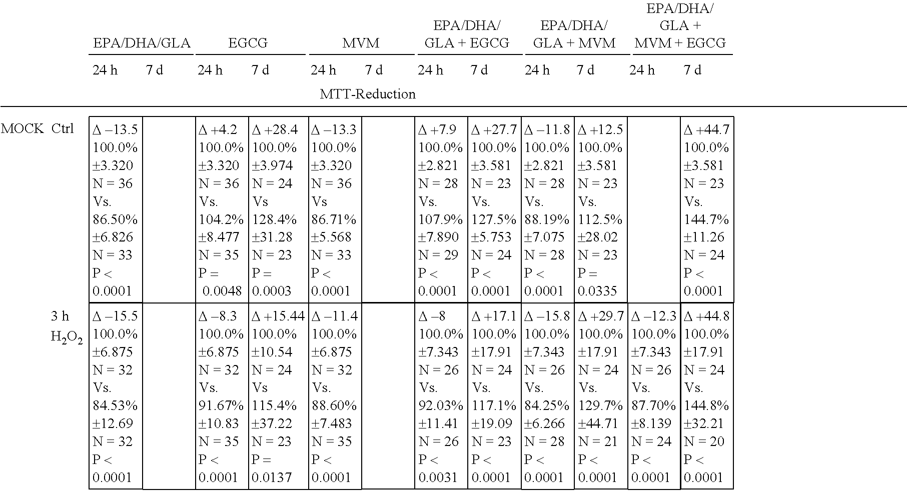

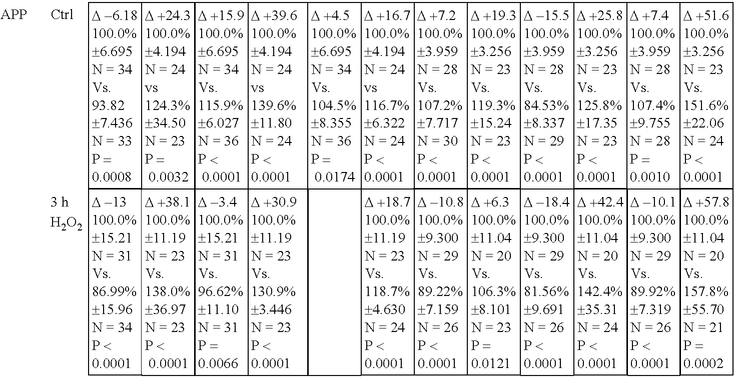

Color code of the table: white boxes: significant negative impact; gray boxes: significant positive results; empty boxes: no significant results.
Signitificant positive results are marked with an asterisk: *
Mock Ctrl (untreated control)=100%
Mock 3h H2O2=100% (also normalized to 100% with regard to protective effects in the presence of H2O2)
APP Ctrl (untreated control)=100%
APP 3h H2O2=100% (also normalized to 100% with regard to protective effects in the presence of H2O2)
Δvalue=% difference value to 100% of corresponding control value (untreated control or H2O2 treated control)
Means, ±SD, paired student's t-test

1. A composition for use in a method of preventing and/or treating and/or reducing organic, including symptomatic, mental disorders, comprising:
a) Docosahexaenoic acid (DHA) and/or the pharmaceutically acceptable derivatives and/or precursors thereof;
b) Eicosapentaenoic acid (EPA) and/or the pharmaceutically acceptable derivatives and/or precursors thereof;
c) γ-linolenic acid (GLA) and/or the pharmaceutically acceptable derivatives and/or precursors thereof; and
at least one vitamin selected from the group consisting of vitamin B6, folic acid, vitamin B12, vitamin E, and/or vitamin D, at least one mineral selected from the group consisting of selenium and zinc, and an effective amount of polyphenolic plant extract with antioxidant properties.

2. Composition according to claim 1, for use in a method of preventing and/or treating and/or reducing dementia in Alzheimer's disease, vascular dementia, senile dementia;
other degenerative diseases of the nervous system, such as Alzheimer's disease; or symptoms of age associated cognitive decline; symptoms of subjective cognitive impairment; age related cognitive decline (ARCD); age associated memory impairment (AAMI); or mild cognitive impairment in a subject of at least 50 years old.

3. Composition according to any one of the preceding claims, wherein said composition is in the form of a formulation suitable for oral administration.

4. Composition to any one of the preceding claims, wherein said oral formulation is present in the form of hard or soft capsule, microcapsule, oil, syrup, suspension, solid or semi-solid, tablet, sugar-coated tablets, powder, pellet, film-coated tablet, granule, emulsion, paste, gels, lozenges, gums, or chewable formulation, such as chewing-gum.

5. Composition according to claim 4, wherein said oral formulation is a gastro-resistant formulation or a non gastro-resistant formulation.

6. Composition according to any one of the preceding claims, wherein the subject is a healthy human subject or a human subject in need of such treatment.

7. Composition according to any one of the preceding claims, wherein said composition is administered as a food supplement.

8. Composition according to claim 1, wherein said composition is in the form of a pharmaceutical composition, and comprises a pharmaceutically acceptable vehicle.

9. Composition according to any one of the preceding claims, wherein EPA, DHA and GLA are present in a ratio of approximately 9:3:1.

10. Composition according to claim 1 wherein the vitamin is vitamin B6, the mineral is selenium and the polyphenolic plant extract with antioxidant properties is a green tea extract.

11. Composition according to claim 1, wherein said composition further comprises an antioxidant.

12. Composition according to claim 10, wherein said antioxidant is selected among tocopherols (4 toco), Origanox®, oil extract from oregano (Origanum vulgare), thyme (Thymus vulgaris), rosemary (Rosmarinus officinalis), sage (Salvia officinalis) and clove (Syzygium aromaticum), and/or vitamin C.

13. A method preventing and/or treating and/or reducing organic, including symptomatic, mental disorders, comprising administering to a subject in need thereof a therapeutically effective amount of the composition comprising:
a) eicosapentaenoic acid (EPA)and/or the pharmaceutically acceptable derivatives and/or precursors thereof;
b) docosahexaenoic acid (DHA) and/or the pharmaceutically acceptable derivatives and/or precursors thereof;
c) γ-linolenic acid (GLA) and/or the pharmaceutically acceptable derivatives and/or precursors thereof, and
at least one vitamin selected from the group consisting of vitamin B6, folic acid, vitamin B12, vitamin E, and/or vitamin D, at least one mineral selected from the group consisting of selenium and zinc, and an effective amount of polyphenolic plant extract with antioxidant properties.

14. The method according to claim 13, wherein the therapeutically effective amount of the composition is administered for preventing and/or treating and/or reducing dementia in Alzheimer's disease, vascular dementia, senile dementia; other degenerative diseases of the nervous system, such as Alzheimer's disease; or symptoms of age associated cognitive decline; symptoms of subjective cognitive impairment; age related cognitive decline (ARCD); age associated memory impairment (AAMI); or mild cognitive impairment in a subject of at least 50 years old.

15. The method according to claim 13, wherein the composition is in the form of a formulation suitable for oral administration.

16. The method according to claim 15, wherein the oral composition is in the form of hard or soft capsule, microcapsule, oil, syrup, suspension, solid or semi-solid, tablet, sugar-coated tablets, powder, pellet, film-coated tablet, granule, emulsion, paste, gels, lozenges, gums, or chewable formulation, such as chewing-gum.

17. The method according to claim 16, wherein the oral formulation is a gastro-resistant formulation or a non gastro-resistant formulation.

18. The method according to claim 13, wherein the subject is a healthy human subject or a human subject in need of such treatment.

19. The method according to claim 13, wherein the composition is administered as a food supplement.

20. The method according to claim 13, wherein the composition is in the form of a pharmaceutical composition, and comprises a pharmaceutically acceptable vehicle.

21. The method according to claim 13, wherein EPA, DHA and GLA are present in a ratio of approximately 9:3:1.

22. The method according to claim 13, wherein the vitamin is vitamin B6, the mineral is selenium and the polyphenolic plant extract with antioxidant properties is a green tea extract.
